(19) United States
(12) Patent Application Publication
(10) Pub. No.: US 2006/0235297 A1
(43) Pub. Date: Oct. 19, 2006

(54) Method for imaging lesion and lesion imaging system

(76) Inventor: Yasushi Kawamoto, Kyoto City (JP)

Correspondence Address: GREGORY L BRADLEY;MEDRAD INC, ONE MEDRAD DRIVE, INDIANOLA, PA, 15051, US

(21) Appl. No.: 11/389,692

(22) Filed: Mar. 27, 2006

(30) Foreign Application Priority Data
Nov. 17, 2004 (JP) 2004332756

(51) Int. Cl. A61B 6/00

(52) U.S. Cl. 600/431

(57) ABSTRACT

The invention provides a method for imaging a lesion area that includes a process of infusing a small amount of contrast medium from the syringe for a time Tn and using an image capturing device to measure a change in blood contrast medium concentration, a process of summing data that are based on the change in blood contrast medium concentration during the time Tn over a period of a required infusion time Tc for an amount of contrast medium necessary for diagnosis, to predict the contrast effect during the required infusion time Tc, and a process of displaying the predicted contrast effect and urging determination of whether or not to change the infusion conditions of the contrast medium.

### BACKGROUND OF THE INVENTION

1. Technical Field

The present invention relates to methods for imaging lesions in a patient and lesion imaging systems for creating a diagnostic image of the patient.

2. Description of the Related Art

To create diagnostic images of lesions, conventionally, a contrast medium is infused into a patient and then x-rays are irradiated onto the patient. The contrast medium in such cases is a pharmaceutical agent whose x-ray absorbency is different from that of human tissue, and for example is barium sulfate or an iodine solution. Of these, barium sulfate is administered orally, whereas iodine solutions are frequently used for injection or infusion into the body, but in the following description the contrast medium is not limited to iodine solutions.

FIG. 10 is a block diagram of a lesion imaging system that has been proposed in the past (for example, see JP H07-204176A). This system is for controlling the infusion of contrast medium into a patient 6 from outside an imaging room 4 into which the patient 6 has been led. Inside the imaging room 4 are provided an injector head 3, a head control unit 1, a first infrared transceiver 40, and an image capturing device 2, which is discussed later. Outside the imaging room 4 are arranged a second infrared transceiver 41 and a personal computer (hereinafter, “PC”) 5 that are in serial communication with the first infrared transceiver 40.

As shown in FIG. 11, two syringes 8, 80 linked to a catheter 30 that is connected to the patient 6 are connected to the injector head 3. An infusion needle 31 is attached to the tip portion of the catheter 30. The one syringe 8 contains contrast medium and the other syringe 80 contains saline, and the injector head 3 is provided with a piston that pushes out the liquid within the syringes 8 and 80, and a piston drive actuator (not shown). The head control unit 1 receives signals from the first infrared transceiver 40 and controls the amount of piston movement and which of the syringes 8 or 80 to drive.

When performing a test, first the patient 6 lies on his or her side on a table 21 of the image capturing device 2 and the infusion needle 31 is stuck into the patient 6. The physician or testing technician performing the test on the patient 6 operates the PC 5 and inputs a signal for the infusion of contrast medium from the infusion needle 31. This signal is transferred to the head control unit 1 via the second and first infrared transceivers 41 and 40, and then, first contrast medium is infused into the patient 6 from the one syringe 8. The required infusion time Tc that is necessary to infuse an amount of contrast medium that is sufficient for imaging is found in advance from the extent of the lesion to be imaged. Once contrast medium has been infused for the required infusion time Tc, saline is delivered from the other syringe 80 to flush the catheter 30 and infuse any contrast medium remaining in the catheter 30 into the patient 6. The contrast medium flows toward the lesion through a vein or artery of the patient 6.

The patient 6 is then examined by the image capturing device 2. The image capturing device 2 is a CT (computed tomography) device that as shown in FIG. 12 is provided with a table 21 that can move forward and backward and on which the patient 6 is placed, and a circular opening 22 through which the table 21 passes. An x-ray tube 23 that irradiates x-rays onto the patient 6, and a detector 24 that receives x-rays that have passed through the patient 6 are provided in opposition to one another along the periphery of the opening 22, symmetrically flanking the center of the opening 22 between them, and the x-ray tube 23 and the detector 24 are provided such that they can rotate concentrically with respect to the center of the opening 22.

When the table 21 passes through the opening 22, the x-ray tube 23 and the detector 24 rotate concentrically with respect to the center of the opening 22, irradiating x-rays onto the lesion area. As mentioned above, the contrast medium has a different x-ray absorbency then does body tissue, and thus when the contrast medium has arrived at the lesion, a cross-sectional image of the lesion of the patient 6 is obtained as a monochrome image. The monochrome image is displayed on a display 20 that is connected to the image capturing device 2. The task of irradiating x-rays from the x-ray tube 23 to obtain a sectional image of a lesion area is generally referred to as a “scan.”

Conventionally, the tomographic contrast effect—that is, what level of image resolution or clarity is obtained—from the contrast-medium infusion has often been dictated by how experienced the physician or the testing technician is.

The contrast effect that is obtained can vary according to infusion conditions such as the infusion rate, and according to the physical dimensions of the patient 6, however, and it is difficult to correctly anticipate the contrast effectiveness based on experience alone.

### BRIEF SUMMARY OF THE INVENTION

It is an object of the present invention to find infusion conditions, such as the infusion rate, that are required for a desired contrast effect to be obtained, so as to obtain a more suitable contrast effect.

A method for imaging a lesion area of the present invention includes a process of infusing a small amount of contrast medium from the syringe 8 for a time Tn, and using the image capturing device 2 to measure a change in blood contrast medium concentration in a lesion area of a patient;

a process of summing data that are based on the change in blood contrast medium concentration during the time Tn over a period of a required infusion time Tc for an amount of contrast medium necessary for diagnosis, to predict a contrast effect during the required infusion time Tc; and

a process of displaying the predicted contrast effect and urging determination of whether or not to change the infusion conditions of the contrast medium.

In the present invention, a small amount of contrast medium is infused into a patient in advance for a time Tn and the change in blood concentration of the contrast medium in the lesion area of the patient is measured.

Next, data based on the change in blood concentration of the contrast medium during the time Tn are summed over a required infusion time Tc for an amount of contrast medium necessary for diagnosis in order to predict, that is, simulate, the contrast effect for the required infusion time Tc. The simulated contrast effect is displayed to prompt decision on whether or not to change the infusion conditions, such as the infusion rate, of the contrast medium. The physician or testing technician views the simulated contrast effect, and if an amount of contrast medium that is necessary for testing of the lesion area has been infused into the patient 6, sets these as the desired contrast medium infusion conditions 6.

Thus, it is possible to obtain a more suitable contrast effect, which is necessary for diagnosis.

From the following detailed description in conjunction with the accompanying drawings, the foregoing and other objects, features, aspects and advantages of the present invention will become readily apparent to those skilled in the art.

### BRIEF DESCRIPTION OF THE SEVERAL VIEWS OF THE DRAWINGS

FIG. 1 is a block diagram of the lesion imaging system.

FIG. 2 is a block diagram of the head control unit interior.

FIG. 3 is a plan view showing the screen of the PC.

FIG. 4 is a graph plotting the contrast medium infusion rate versus time for the test bolus.

FIG. 5 is a graph plotting the relationship between the CT value of the image and time.

FIG. 6 is a graph plotting the contrast medium infusion rate set initially versus time.

FIG. 7 is a graph plotting the relationship between the CT value and time when simulating an image from the values of the test bolus.

FIG. 8 is a graph plotting the relationship between the CT value and time when simulating an image from the values of the test bolus.

FIG. 9 is a flowchart showing the procedure of the test bolus and image simulation.

FIG. 10 is a block diagram of a conventional lesion imaging system.

FIG. 11 is a diagram showing the injector head.

FIG. 12 is a perspective view of the image capturing device.

### DETAILED DESCRIPTION OF THE INVENTION

Overall Structure

An example of the present invention is described in detail below using the drawings. This example is characterized in that simply by infusing a small amount of contrast medium in advance it is possible predict the contrast effect when an amount of contrast medium that is required for testing is infused.

FIG. 1 is a block diagram of the lesion imaging system according to this example. An image capturing device 2, which is a CT device furnished with an injector head 3, a head control unit 1, a first infrared transceiver 40 and a display 20, is arranged inside an imaging room 4. A second infrared transceiver 41 and a PC 5 in serial communication with the first infrared transceiver 40 are arranged outside the imaging room 4. The PC 5 is provided with a screen 50 and an operation button 51, as is the case normally, and a physician or testing technician inputs the initial infusion conditions for the contrast medium, such as the infusion rate, through the PC 5. The injector head 3 has the same structure as that of the conventional example depicted in FIG. 11.

Although the structure of this example resembles the conventional structure shown in FIG. 10, it is different in that the image capturing device 2 is linked to the head control unit 1. It should be noted that providing the first and second infrared transceivers 40 and 41 allows the person performing testing of the patient 6 to remotely operate the injector head 3. However, in lieu of this it is also possible to directly connect the PC 5 and the head control unit 1 to one another without providing the first and second infrared transceivers 40 and 41.

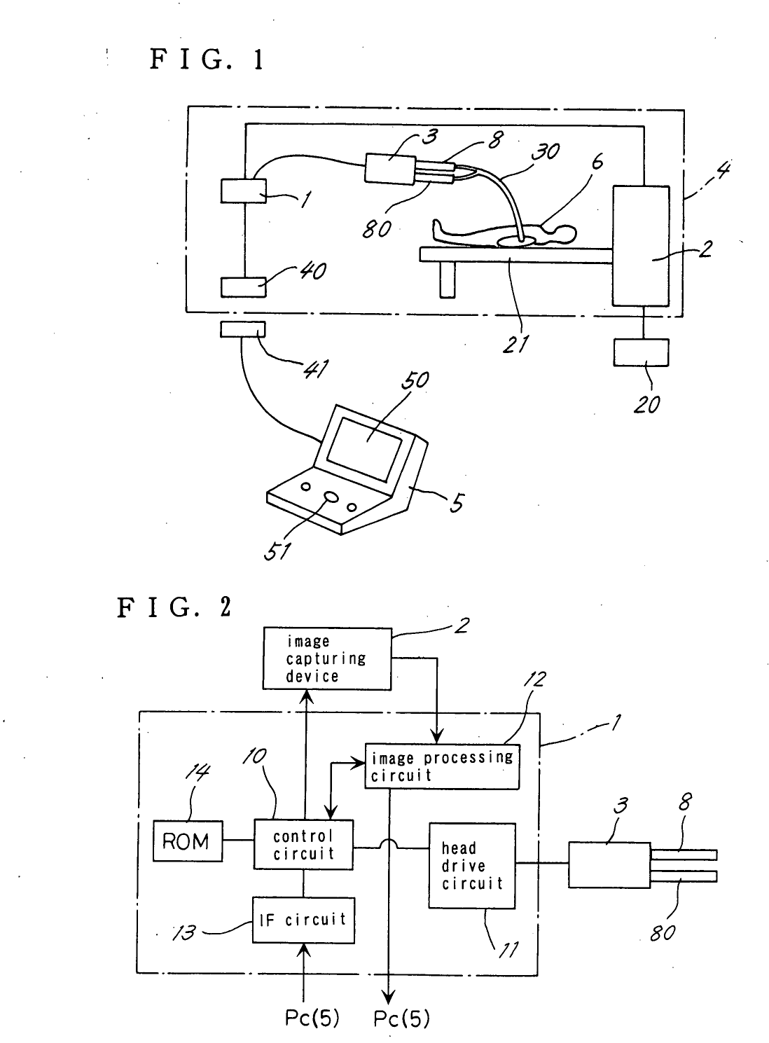

FIG. 2 is a block diagram of the internal structure of the head control unit 1. An operation signal from the PC 5 is input to a control circuit 10 via an interface (IF) circuit 13. The control circuit 10 is a microcomputer that has a timer function of dividing an internal operation clock. The control circuit 10 is connected to a head drive circuit 11 that is linked to the injector head 3, and to a RAM 14 that can store the white level of the image from the image capturing device 2, a time Tc required to infuse an amount of contrast medium necessary for diagnosis and a time Tn for infusing a small amount of contrast medium, which are discussed later. The time Tc necessary to infuse an amount of contrast medium required for diagnosis is input in advance by the physician, for example.

The signal from the control circuit 10 is also sent to the image capturing device 2, and the brightness of the image from the image capturing device 2 is detected by an image processing circuit 12 and its white level is input to the control circuit 10.

It should be noted that the white level of the captured image is shown by a value called the CT value. The CT value is a dimensionless value that is low for regions through which x-rays pass easily, such as air, and high for regions through which x-rays do not pass easily. More specifically, the CT value is −1000 if x-rays are emitted into air, 0 if emitted into water, from 200 to 500 if emitted into human bone, and from 50 to 1000 if emitted into a contrast medium. Consequently, when the CT value of the image obtained from the image capturing device 2 reaches 50 or more, then it is understood that the contrast medium has begun to arrive at the lesion.

FIG. 3 is a plan view showing the screen 50 of the PC 5. An image of the syringes 8 and 80 that can be lit up is depicted on the screen 50, and the image of the syringe 8 lights up during contrast medium infusion and the image of the syringe 80 lights up during saline infusion. The screen 50 shows the infusion rate, the amount left to be infused, and the infusion time of the contrast medium or the saline. The person performing the testing operates the operation button 51 of the PC 5 to select the desired infusion rate, for example.

Method for Imaging Lesion

Test Bolus

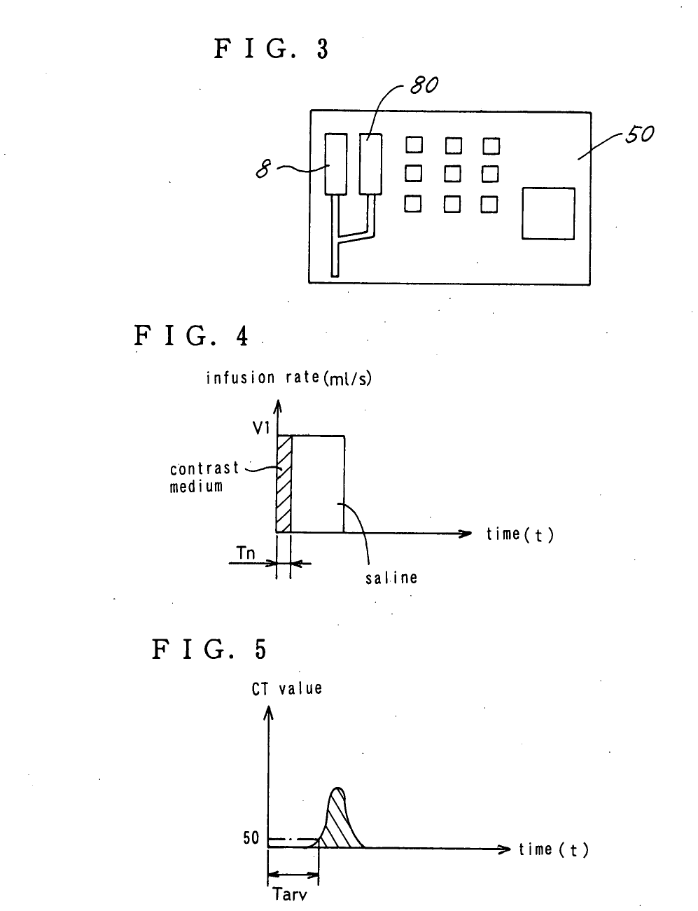

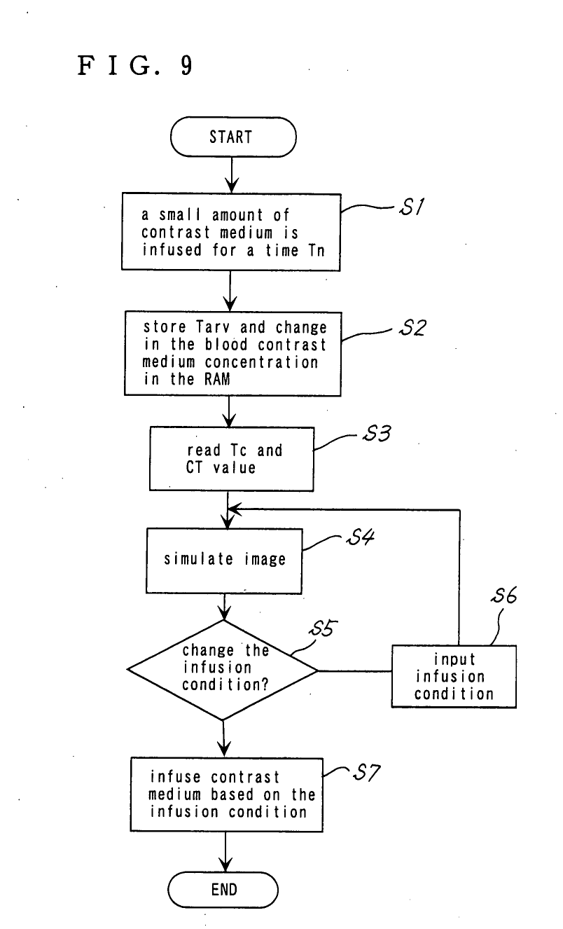

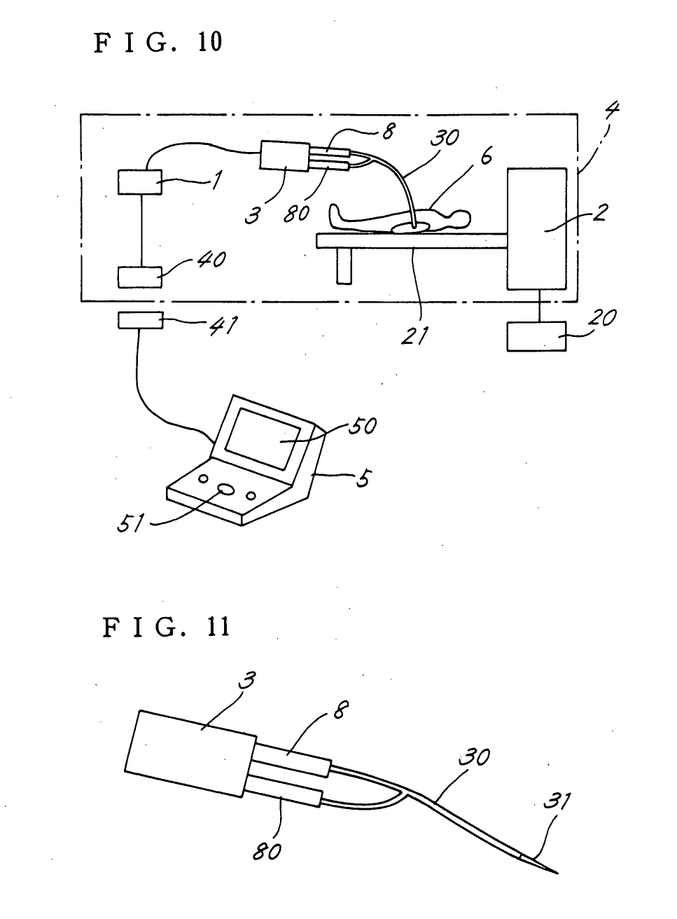

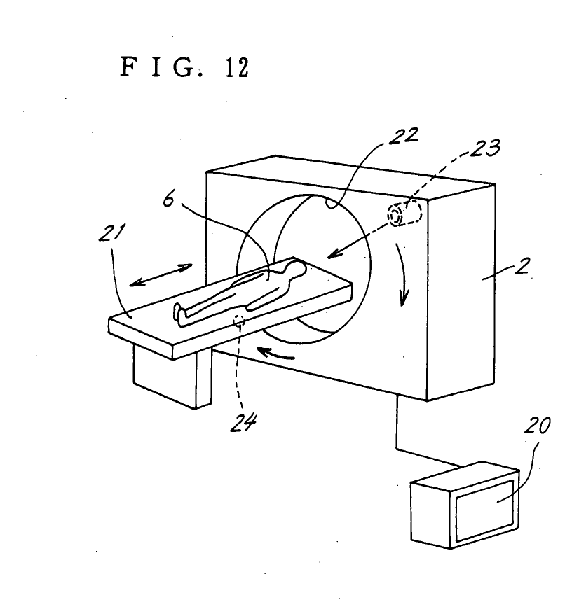

The method of imaging a lesion area is described below using the graphs of FIG. 4 to FIG. 8 and the flowchart of FIG. 9. This example has the characteristic that first a small, detectable amount of contrast medium is infused.

An infusion needle 31 is inserted into the patient 6 and the patient 6 is led into the image capturing device 2. As the table 21 moves, the x-ray tube 23 emits x-rays into the lesion area of the patient 6. The person performing this testing of the patient 6 operates the PC 5 to start testing. A signal ordering the start of testing is delivered to the head control unit 1 via the second and first infrared transceivers 41 and 40, and the control circuit 10 receives this signal from the PC 5 and activates the head drive circuit 11. As shown in FIG. 4, a small amount of contrast medium on the order of 1 to 20 mL is infused into the patient 6 from the syringe 8 for a time Tn of two or three seconds, for example (S1). The infusion rate at this time is a velocity V1 selected on the screen of the PC 5 and is on the order of about 10 mL per second, but this is not a limitation. After the contrast medium has been infused, then 10 to 30 mL of saline are infused from the other syringe 80 to completely infuse the contrast medium remaining in the catheter 30.

When infusion of the small amount of contrast medium is started, the control circuit 10 activates an internal timer and transmits a signal to the image capturing device 2 indicating that contrast medium has been infused. The image capturing device 2 displays the image obtained by scanning on the display 20 and transmits every unit image, for example, every frame, to the image processing circuit 12. Because the x-ray tube 23 is emitting x-rays into the lesion area, the lesion appears white when the contrast medium arrives at the lesion.

The concentration of contrast medium in the lesion area is proportional to the CT value, which is the white level of the captured image, and is found from the CT value. In other words, it can be understood that the contrast medium has begun to arrive at the lesion when the CT value of the image that is received exceeds a fixed value. The control circuit 10 obtains a time Tarv (see FIG. 5) from the start of infusion until the CT value exceeds a fixed value, and more specifically, until the CT value exceeds 50.

The image processing circuit 12 measures the CT value every unit time, for example, every frame, and transmits this to the control circuit 10. The control circuit 10 stores the change in the blood contrast medium concentration, which is indicated by the value of the time Tarv and the CT value, in the RAM 14 (S2). That is, the image information shown by the diagonal hatching in FIG. 5 is stored in the RAM 14. The operation of infusing a small amount of contrast medium for the time Tn and finding the change in blood concentration of the contrast medium is referred to as a “test bolus,” with “bolus” being a term that means a single infusion.

Contrast Effect Simulation

Next, because the time Tc required for infusion of contrast medium in an amount necessary for diagnosis has been stored in the RAM 14 in advance as a known value, the control circuit 10 reads the required infusion time Tc from the RAM. It also reads the CT value for each unit time from the RAM 14 (S3). Additionally, it also reads the initial infusion conditions of the contrast medium for diagnosing the lesion, which have been input to the PC 5. These initial infusion conditions are obtained based on the experience of the physician, for example.

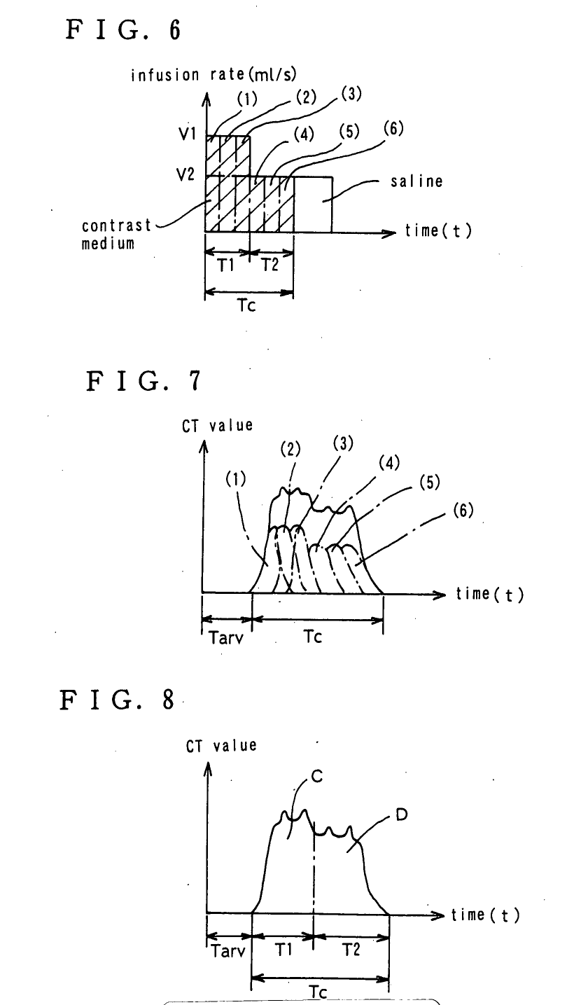

Take a case in which the initial infusion conditions are those shown in FIG. 6, in which infusion is continued at the same velocity V1 as that of the test bolus for a time T1 and then the infusion rate is lowered to V2 and infusion is performed for a time T2. The time T1 is longer than the time Tn, and the time Tc is the sum of time T1 and time T2.

The control circuit 10 sums the data of the CT value of the test bolus for the time Tn, that is, the image information shown by the diagonal hatching in FIG. 5, during the period of time T1 and transmits this to the image processing circuit 12. The image processing circuit 12 draws an image C corresponding to the time T1 of the graph of FIG. 6 once the time Tarv has elapsed from the start of infusion (see FIG. 8). If the time T1 is three times the time Tn ((1), (2), and (3) in FIG. 6), then the image processing circuit 12 treats this as if the test bolus has been performed consecutively three times. The image information obtained based on the test bolus is summed three times over the time T1, simulating the image C corresponding to the time T1 and drawing it on the screen of the PC 5 ((1), (2), and (3) in FIG. 7) (S4).

Next, the control circuit 10 predicts the CT values at the infusion rate V2 from the data of the CT values of the test bolus. Since the infusion rate V2 is not as fast as the infusion rate V1, the CT values at the velocity V2 should be lower than the CT values at the velocity V1. The CT values at the infusion rate V1 are known from the test bolus, and thus the control circuit 10 sets the CT values to a lower level that corresponds to the infusion rate V2. These CT values are summed over the period of the time T2 and transmitted to the image processing circuit 12. In a case where the time T2 is three times the time Tn ((4), (5), and (6) in FIG. 6), the image processing circuit 12 obtains CT values of a low level that correspond to the infusion rate V2, sums these over the time T2, and in continuation of the image C draws an image D that corresponds to the time T2 of the graph of FIG. 6 on the screen of the PC 5 (see FIG. 8). In other words, a simulated image for the time Tc is displayed on the screen of the PC 5. A display urging input on whether or not to change the infusion conditions of the contrast medium, such as the infusion rate, is displayed on the PC 5 (S5). The physician or testing technician determines whether or not to change the infusion conditions of the contrast medium based on the results of this simulation. That is, if he determines that the simulated image has a resolution or brightness that is suitable for diagnosis, then he makes an input to not change the infusion conditions to PC 5. If he determines that the simulated image is insufficiently clear for diagnosis, then he makes an input to the PC 5 to raise the infusion rate (S6). The image is simulated again in the event that the infusion rate is changed.

The contrast medium is subsequently infused for the time Tc based on the infusion conditions that have been entered. After the contrast medium has been infused, then, as mentioned above, saline is infused to completely infuse any contrast medium remaining in the catheter 30.

If the same lesion area of the same patient 6 is to be tested repeatedly, it is a nuisance to have to perform a test bolus each time. Also, the blood concentration of the contrast medium does not vary significantly for the same lesion area of the same patient 6. Accordingly, in such a case, the physician or testing technician performing the test can read the CT values for the time Tn from the RAM 14 to simulate the captured image for the time Tn and then determine whether or not to change the infusion conditions of the contrast medium.

In this embodiment, a predetermined small amount of contrast medium is infused into the patient for a time Tn and the change in concentration of contrast medium in the blood at the lesion area of the patient is measured.

Next, the contrast effect for the required infusion time Tc is estimated, that is, simulated, by summing the data based on the change in blood concentration of the contrast medium during the time Tn over the required infusion time for an amount of contrast medium necessary for diagnosis. The simulated contrast effect is displayed to prompt a decision on whether or not to change the infusion conditions, such as the infusion rate, of the contrast medium. The physician or testing technician views the simulated contrast effect, and if an amount of contrast medium necessary to examine the lesion has been infused, sets these as the desired contrast medium infusion conditions.

It is thus possible to obtain a more appropriate contrast effect required for diagnosis.

In the above discussion, a CT device served as an illustrative example of the device for capturing an image of a lesion area, but in place of this it is also possible to use a MRI (magnetic resonance imaging) device. It is common knowledge that instead of emitting x-rays, an MRI device emits a powerful magnetic field and electromagnetic waves to obtain a magnetic resonance image.

Only selected embodiments have been chosen to illustrate the present invention. To those skilled in the art, however, it will be apparent from the foregoing disclosure that various changes and modifications can be made herein without departing from the scope of the invention as defined in the appended claims. Furthermore, the foregoing description of the embodiments according to the present invention is provided for illustration only, and not for limiting the invention as defined by the appended claims and their equivalents.

What is claimed is:

1. A lesion-imaging method using a system furnished with a syringe through which contrast medium is introduced, a control unit for operating the syringe to control contrast-medium infusion quantity, and a device for capturing images of a lesion area in a patient into whom contrast medium has been infused through the syringe, the method comprising:
a step of infusing a small amount of contrast medium from the syringe for a time Tₙ, and, using the image-capturing device, measuring changes in blood contrast-medium concentration in the patient's lesion area;
a step of summing, over a period T_{c}, required to infuse a diagnostic-effective amount of contrast medium, data based on the change in blood contrast-medium concentration during the time Tₙ, to predict contrast effect for the period T_{c}; and
a step of displaying the predicted contrast effect and prompting determination of whether to change the contrast-medium infusion conditions.

2. The lesion-imaging method according to claim 1, further comprising:
a step of infusing saline after the small amount of contrast medium has been infused from the syringe.

3. A legion imaging system comprising a syringe through which contrast medium is introduced, a control unit for controlling the quantity of contrast medium infused through the syringe, and a device for capturing images of a lesion area in a patient into whom contrast medium has been infused through the syringe, wherein the control unit stores a program for:
infusing a small amount of contrast medium from the syringe for a time Tₙ;
using the image-capturing device, measuring changes in blood contrast-medium concentration;
summing, over a period T_{c}, required to infuse a diagnostic-effective amount of contrast medium, data based on the change in blood contrast-medium concentration during the time Tₙ, to predict contrast effect for the period T_{c};
displaying the predicted contrast effect and prompting determination of whether to change the contrast-medium infusion conditions.
